(19) United States
(12) Patent Application Publication
(10) Pub. No.: US 2022/0072040 A1
(43) Pub. Date: Mar. 10, 2022

(54) TREATMENT METHOD FOR GRAFT-VERSUS-HOST DISEASE

(71) Applicant: REGENEX LLC, Moscow (RU)

(72) Inventor: Svetlana N. BYKOVSKAIA, Woodland Hills, CA (US)

(73) Assignee: REGENEX LLC, Moscow (RU)

(21) Appl. No.: 17/314,997

(22) PCT Filed: Nov. 11, 2019
(86) PCT No.: PCT/IB19/59675

§ 371 Date: May 7, 2021

Related U.S. Application Data
(60) Provisional application No. 62/758,420, filed on Nov. 9, 2018.

(51) Int. Cl. A61K 35/17; C12N 5/0783

(52) U.S. Cl. CPC A61K 35/17; C12N 5/0637

(57) ABSTRACT

Disclosed is a method for treating or preventing GvHD in a transplant patient by administering to the patient autologous, ex vivo-modified and expanded CD4⁺CD25⁺Foxp3⁺CD127^{low} T reg cells.

### PRIORITY CLAIM

This application claims priority to U.S. Provisional application No. 62/758,420, filed Nov. 9, 2018, the contents of which are hereby incorporated herein in their entirety.

### FIELD OF INVENTION

The invention relates to medicine, immunology, and more specifically to the prevention and/or treatment of graft-versus-host disease.

### BACKGROUND

Graft-versus-host disease (GvHD) is an autoimmune disorder which develops following the transplantation of tissue from a genetically different person. GvHD is associated with solid organ transplants and can occur after a blood transfusion if the blood products used have not been irradiated or treated with an approved pathogen reduction system.

GvHD occurs when white blood cells present in the transplanted donor tissue recognize the recipient as foreign and attack the recipient's body. GvHD can also occur after a blood transfusion if the blood products used have not been irradiated or treated with a pathogen reduction system.

Acute GvHD occurs soon after transplant, while chronic HvHD begins after day 100 post-transplant. Acute GvHD is characterized by selective damage to the liver, skin, (rash), mucosa, and the gastrointestinal tract, as well as the immune system (the hematopoietic system, e.g., the bone marrow and the thymus) itself, and the lungs in the form of immune-mediated pneumonitis. Chronic GvHD can also cause damage to connective tissue and exocrine glands over the long term.

GvHD also s associated with stem cell transplants such as those that occur with bone marrow transplants. After bone marrow transplantation, T cells present in the graft, either as contaminants or intentionally introduced into the host, attack the tissues of the transplant recipient after perceiving antigens in the host tissues as antigenically foreign. The T cells produce an excess of cytokines, including TNF-α and interferon-gamma (IFN∛).

A wide range of host antigens can initiate GvHD, including the human leukocyte antigens (HLA). The HLA's most responsible for graft loss are HLA-DR (first six months), HLA-B (first two years), and HLA-A (long-term survival). However, GvHD can occur even when HLA-identical siblings are the donors. HLA-identical siblings or HLA-identical unrelated donors often have genetically different proteins, or minor histocompatibility antigens, that can be presented by Major histocompatibility complex (MHC) molecules to the donor's T-cells, which see these antigens as foreign and so mount an immune response.

GvHD can largely be avoided by performing a T-cell-depleted bone marrow transplant. Unfortunately, while donor T-cells are undesirable as effector cells of GvHD, they prevent the recipient's immune system from rejecting the transplanted graft (host-versus-graft). In addition, as bone marrow transplantation is frequently used to treat leukemias, donor T-cells are important to provide an offensive graft-versus-tumor effect.

Intravenously administered glucocorticoids, such as prednisone, are the standard of care in acute GvHD and chronic GVHD. These glucocorticoids suppress the T-cell-mediated immune response to the host tissues. However, this immune-suppression raises the risk of infections and cancer relapse.

Thus, what is needed are effective compositions and methods of treating GvHD.

### SUMMARY

It has been discovered that there is an inverse relationship between the number of T reg cells in the peripheral blood of patients with GvHD and the stage or degree of rejection. This discovery has been exploited to develop the present method of treating GvHD. Administering autologous, ex vivo-expanded CD4⁺CD25⁺Foxp3⁺CD27^{low} regulatory T (T reg) cells starting at the first week or second week after transplant lowers the level of autoimmune activity of certain T cells, thereby reducing symptoms of rejection and maintaining the acceptance of the recipient tissues indefinitely or for at least longer periods of time than what is seen on average without treatment.

In one aspect, the disclosure provides a method of preventing or treating GvHD in a patient in need thereof, comprising: administering a therapeutically effective amount of autologous, modified and expanded T reg cells to the patient starting 1-2 weeks after transplant.

In some embodiments, the administering step comprises administering the therapeutically effective amount of autologous modified and expanded T reg cells more than one time after the start of treatment to increase the number of T reg cells in patient's blood to a number comparable to number in the blood of healthy donors. In some embodiments, the administering step comprises administering a therapeutically effect amount of these T reg cells about 1 to 7 times at the start of treatment.

In some embodiments the method further comprising measuring the number of T reg cells in the blood of the patient before and after each administering step. In particular embodiments, the number of T reg cells in a patient's blood is measured weekly, or every 1 to 3 months, or every 2 to 3 months, after the initial administering step.

In particular embodiments, the method further comprises a second administering step if the number of T reg cells measured in the peripherical blood of a patient after the first administering step is less than the number of T reg levels in blood of a healthy donor.

In some embodiments, the autologous T reg cells administered are expanded ex vivo before they are administered to the patient. In certain embodiments, the number of autologous modified and expanded T reg cells administered at one time is about 1×10⁶ to about 1.1×10⁷ per kg body weight to the patient. In many embodiments, the T reg cells are administered by subcutaneous, intravenous, and/or intramuscular injection.

Also provided by the present disclosure is a method of inhibiting the activity of autoimmune, autologous, cytotoxic T and B cells in a patient suffering from GvHD, comprising administering a therapeutically effective amount of autologous, modified and expanded T reg cells to the patient. In some embodiments, the administering step is performed more than one time throughout the life of the patient. In certain embodiments, the administering step is performed weekly, and/or every 1 to 7, 2 to 6, 3 to 6, or 4 to 5 months after the first administering step.

In some embodiments, the administering step comprises administering about 1×10⁶ to about 1.1×10⁷ autologous modified and expanded T reg cells per kg body weight to the patient. In many embodiments, the T reg cells are administered by subcutaneous, intravenous, and/or intramuscular injection.

### DESCRIPTION OF THE FIGURES

The foregoing and other objects of the present disclosure, the various features thereof, as well as the invention itself may be more fully understood from the following description, when read together with the accompanying drawings in which:

FIG. 1A is a scatter plot showing characteristic T reg markers in thepopulation of donor mononuclear cells which are CD25⁺;

FIG. 1B is a scatter plot showing characteristic T reg markers in the population of donor mononuclear cells which areFoxp3;

FIG. 1C is a scatter plot showing characteristic T reg markers in the population of donor mononuclear cells which are CD27^{low};

FIG. 2A is a scatter plot showing characteristic T reg markers in the population of donor CD4⁺ cells which are CD25⁺;

FIG. 2B is a scatter plot showing characteristic T reg markers in the population of donor CD4⁺ T cells which are Foxp3;

FIG. 2C is a scatter plot showing characteristic T reg markers in the population of Donor CD4⁺ T cells which are CD27^{low};

FIG. 3A is a scatter plot showing the expression of CD25^{hi} on CD4⁺ T cells after 6 days of cultivation;

FIG. 3B is a scatter plot showing the expression of Foxp3⁺ on CD4⁺ T cells after 6 days of cultivation;

FIG. 3C is a scatter plot showing the expression of CD27^{low} on CD⁺ T cells after 6 days of cultivation; and

FIG. 4 is a graphic representation showing the increase in total number of cells (gray columns) and the increase in the number of T reg cells (shaded columns) at different stages of cultivation.

### DETAILED DESCRIPTION

Throughout this application, various patents, patent applications, and publications are referenced. The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

It has been discovered that the number of T reg cells in a patient suffering from GvHD variable, and that there is an inverse relationship between the number of T reg cells in the peripheral blood of such patients and the degree of GvHD. This can be determined by studying both the degree of manifestation of the disease process and the number of T reg cells in a same group of patients suffering from GvH, and these not suffering from GvHD after transplant. Thus, a reduced level and functional activity of T reg cells are now understood to play an important role in the progression of the disease. On this basis, a method for treatment of GvHD was developed which includes the immune correction therapy comprising autologous, modified and expanded T reg cells. In addition, it has been determined that treatment with autologous T reg cells can inhibit the activity of autoimmune, autologous, cytotoxic T and B cells in a patient suffering from GvHD.

### Definitions

For convenience, certain terms employed in the specification, examples, and appended claims are collected here. Unless defined otherwise, all technical and scientific terms used herein have the same meaning as commonly understood by one of ordinary skill in the art to which this invention belongs. The initial definition provided for a group or term herein applies to that group or term throughout the present specification individually or as part of another group, unless otherwise indicated.

The articles “a” and “an” are used herein to refer to one or to more than one (i.e., to at least one) of the grammatical object of the article. By way of example, “an element” means one element or more than one element.

The term “or” is used herein to mean, and is used interchangeably with, the term “and/or,” unless context clearly indicates otherwise.

The term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 20%.

As used herein, the term “T reg cells” refers to regulatory T cells with markers CD4⁺CD25⁺Foxp3⁺CD27^{low}.

As used herein, the term “treating” refers to reducing or alleviating the symptoms, and/or preventing the progression of GvHD.

The term “preventing” refers to inhibiting or stopping rejection of recipient tissues in a person affected with GvHD.

The term “native” refers to cells from the body that have not been cultured, modified, expanded, or treated with any compound other than a life-sustaining medium.

“Ex vivo-modified and expanded” refers to native cells removed from the body and treated such that they are modified relative to native T reg cells, and cultivated to proliferate. In the methods of the present disclosure, when cells treated in this way are returned to the body of the patient from which they were originally removed, they are referred to as “autologous, ex vivo-modified and expanded cells”.

As used herein, the term “healthy donor” refers to a mammal, such a human, of the same specie as the patient and who does not have GvHD, does not have any blood-related inflammatory disorder, and is considered by a physician to be in good health. The number of T reg cells in the blood of a healthy donor is used herein to determine the number of T reg cells that are administered to the patient.

### 2. Preparation of Autologous, Ex Vivo—Modified and Expanded T Reg Cells

Autologous, modified and expanded T reg cells administered to a patient with GvHD according to the method of the disclosure are derived from the peripheral blood of the patient. A sample of blood is removed and the fractionated to obtain a mononuclear cell (MNC) fraction from which the T reg cells are later isolated. The MNC fraction can be obtained from blood by any known method of blood fractiona-tion. For example, density gradient centrifugation, e.g. Ficoll-Hypaque density gradient centrifugation, can be used which takes advantage of the density differences between MNC's and other elements found in the blood sample. MNC's and platelets collect on top of the Ficoll-Hypaque layer because they have a lower density than red blood cells and granulocytes which collect at the bottom of the Ficoll-Hypaque layer.

To obtain T cells with a regulatory function, cells in this mononuclear fraction can be exposed to antibodies specific for CD4, as such T cells test positive for this marker. CD4+ cells can then be separated from the MNC fraction, for example, using CD4 MicroBead columns (Myltenyi Biotec, Germany) exposed to a magnetic field. These CD4+cells are then screened for various other surface markers (CD25, Foxp3, and CD127^{low}) which are characteristic of T reg cells, for example, by antibody staining, as described above and in Example 1B below.

The selected T reg cells are then cultivated in a medium with various factors to induce modification, and are expanded, ex vivo. For example, cultivation can be done by growing cells in a growth medium adapted for T cells (e.g., RMP1-1640) after the cells have been stimulated to proliferate (e.g., by exposure to allogenic, antigen producing cells treated with mitomycin C, or TGF-B1 and IL-2).

To determine if the ex vivo-modified and expanded T reg cells have comparable suppressor activity relative to the native T reg cells (circulating in blood), these cells are tested for their ability to suppress the proliferation of certain target cells involved in the autoimmune process. This can be done using any assay involving contacting certain selected target cells (e.g., those in a mixed lymphocyte sample) with the expanded T reg cells, and measuring the target cell's ability to proliferate.

### 3. Pharmaceutical Formulation

To prepare the pharmaceutical composition, the ex vivo-modified and expanded T reg cells having suppressor activity are suspended in a pharmaceutically acceptable carrier. This can be accomplished, e.g. by washing them twice in PBS, centrifuging them, and suspending the cell pellet in the carrier.

The phrase “pharmaceutically acceptable carrier” is employed herein to referto liquid solutions which are, within the scope of sound medical judgment, suitable for use in contact with the live T reg cells without affecting their activity, and without being toxic to the tissues of human beings and animals or causing irritation, allergic response, or other complications, commensurate with a reasonable benefit/risk ratio. A useful pharmaceutically acceptable carrier may be an injectable solution which is biocompatible with the T reg cells and does not reduce their activity or cause their death.

Non-limiting examples of materials which can serve as pharmaceutically acceptable carriers include a solvent or dispersion medium containing, for example, sterile intravenous glucose/dextrose sugar solutions, Ringer's lactate or compound sodium lactate solution.

Prevention of the action of microorganisms can be achieved by various antibacterial and antifungal agents, for example Clindamycin, Fluconazole, and/or Amphotericin B. Sterile injectable solutions of expanded T reg cells can be prepared by incorporating the live cells in the required amount in anappropriate carrier.

The number of T reg cells which can be combined with a carrier material to produce a single-dosage form will vary depending upon the subject being treated, the particular mode of administration, and/ or the degree of GvHD among others. Ultimately, the number of T reg cells in the pharmaceutical composition is that number that causes a therapeutic effect when administered to the patient. For example, the effective amount of the autologous, modified and expanded T reg cells may be about 1×10 to about 1.1×10⁷ T reg cells/kg body weight, about 2×10 to about 8×10 T reg cells/kg body weight, about 4×10 to about 7×10 T reg cells/kg body weight, or about 5×10 to about 7×10 T reg cells/kg body weight.

### 4. Therapeutic Administration

Administration of the formulation containing the autologous T reg cells is useful to prevent or treat and/or to inhibit the activity of autoimmune, autologous, cytotoxic T and B cells in a patient suffering from graft-versus-host disease. This step comprises administering a therapeutically effective amount of autologous, modified and expanded T reg cells to the patient.

Methods of administration of the T reg cells in the pharmaceutical composition according to the disclosure described herein can be by any of a number of methods well known in the art. These methods include systemic or local administration by injection. Exemplary routes of administration include intravenous, intramuscular, intraperitoneal, or subcutaneous injection, and any combinations thereof.

The initial administering step may be a single administration strategy one or two weeks after transplant, or may comprise multiple administrations every 1, 2, or 2-4 weeks after the initial administration. The initial administrating steps may be performed every 1 to 8 weeks. The number of initial administering steps at the start of treatment depends on the initial level of T reg cells in a patient's blood. The goal is to increase T reg cell number in the patient's peripheral blood until it is at the level of a healthy donor. This is determined by measuring the number of T reg cells in the peripheral blood of the patient before and after administering the modified and expanded T reg cells, and comparing that number to the number of T reg cells in the peripheral of a healthy patient. At the start of therapy, 1 to 8, 2 to 7, 3 to 6, 4 to 6, or 3 to 5 T reg cell injections can be administered every 1, 2, to 4 or 3 to 4 weeks.

In addition, in some cases, an additional administering step may be performed every 3 to 6 months after the start of treatment, or at the end of the administration of the initial multiple treatments. However, a physician may determine that administration of the autologous T reg cells may be daily, weekly, or monthly.

In order to determine the number of T reg cells in a transplant patient's blood, a sample is taken for measurement. Any method that enables the measurement of the number of T reg cells can be used. For example, the flow cytometry analysis can be performed. Measurement of the number of T reg cells can be done before and after each initial and secondary administering step(s), and further, can be done months after the initial; and any secondary administering step(s), for example, every two months. The T reg cell-containing pharmaceutical composition can also be administered as part of a combination therapy with other agents to prevent or treat graft-versus-host disease.

“Combination therapy” refers to any form of administration combining two or more different therapeutic compounds, where the second compound is administered while the previously administered T reg cells are still effective in the body (e.g., the two therapeutics are simultaneously effective in the patient, which may include synergistic effects of the two compounds). For example, the different therapeutic compounds can be administered in separate formulations, either simultaneously or sequentially. Thus, a patient who receives such treatment can have a combined (conjoint) effect of different therapeutic compounds.

The following examples provide specific exemplary methods of the invention, and are not to be construed as limiting the invention to their content.

### EXAMPLES

### Example 1

### Isolation and Ex Vivo—Expansion of CD4⁺CD25⁺Foxp3⁺CD27^{low} T reg Cells

All the manipulations are performed under aseptic conditions in a Laminar Flow Class II Biosafety Cabinet which is located in a sterile clean room following to GMP regulations.

### A. Blood Drawing

Peripheral blood (40 ml-50 ml) was taken from the ulnar vein of patients and placed into sterile (Vacutainer, BD, USA). 20 ml-30 ml blood (vacutainer glass serum tubes) was kept at room temperature (RT) for 2 hours, and then centrifuged at 350 g for 15 min. The supernatant was collected into sterile tubes (Falcon, 15 ml conical tubes), which were incubated for 40 min at 56° C. to inactivate complement. The serum was bottled in 1.5 ml vials (Coming, USA) and frozen at −20° C.

### B. Isolation of MNC's

The blood was transferred from tubes with the anticoagulant into 50 ml tubes, dilute 1:1 with Phosphate-buffered Saline (PBS, Ca⁺² Mg ⁺² free, Gibco, United Kingdom). In order to separate lymphocytes, 35 ml MNC suspension was layered over 15 ml of a gradient solution (LimphoSep, d=1.077 g/ml, MP Biomedicals, USA) in 50 ml conical tubes (Falcon, USA). The tubes were centrifuged at 400 g for 30 min at 20° C. The upper layer was aspirated off, leaving the MNC layer, which was transferred to new 50 ml conical tubes. The tubes were filled with buffer and centrifuged at 300 g for 10 min. The cell pellet was resuspended in 50 ml PBS, combined in one tube, and then centrifuged at 300 g, 20° C. for 10 min. This procedure was repeated, and the cell pellet was resuspended in an appropriate amount of buffer.

For an estimation of initial CD4⁺CD25⁺Foxp3⁺CD27^{low} reg cell numbers, the MNC population was stained with anti-CD4⁺, anti-CD25⁺, anti-Foxp3⁺, and anti-CD127⁺ mAbs (Miltenyi Biotec,Germany; eBioscience, USA). The cells were detected by flow cytometry using a MACsQuant (Miltenyi Biotec, Germany).

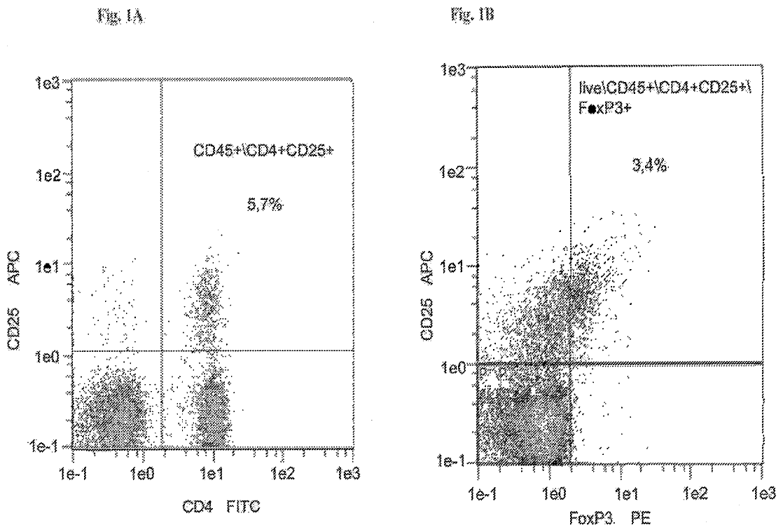

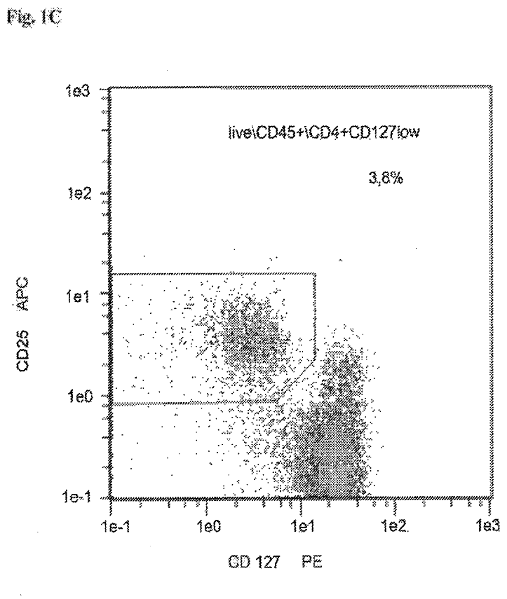

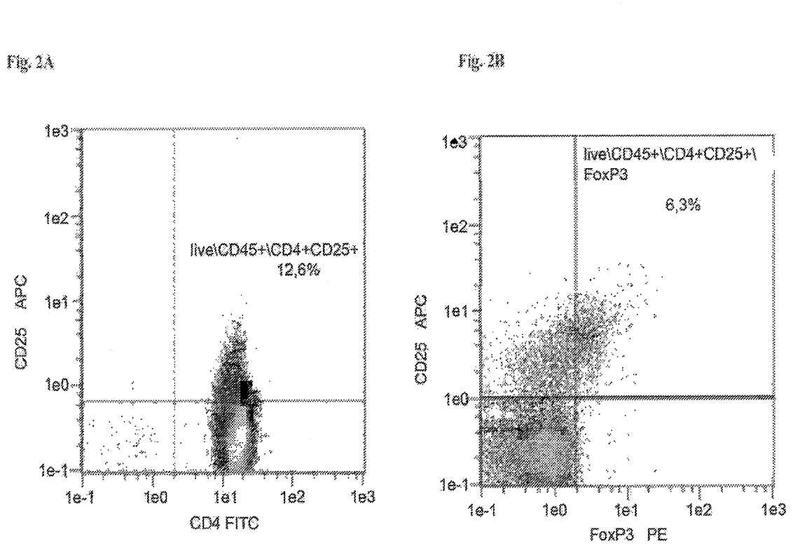

FIGS. 1A-1C show a representative example of characteristic T reg cell markers in the donor's MNCs: 5.7% of CD4⁺ T cells co-expressed CD25⁺ (FIG. 1A); 3.4% of CD4⁺ T cells co expressed Foxp3 (FIG. 1B); and 3.8% of CD4⁺ T cells co-expressed CD127^{low} (FIG. 1C).

### C. Isolation of CD4⁺ T Cells

In order to isolate CD4+ T cells, MNC were magnetically labeled with CD4+ mAbs according to the MACS Miltenyi Biotec (Germany) procedure. The immune phenotype of isolated CD4⁺ T cells was estimated by flow cytometry. In average, 94±4% (n=19) of the isolated cells were CD4⁺ T cells.

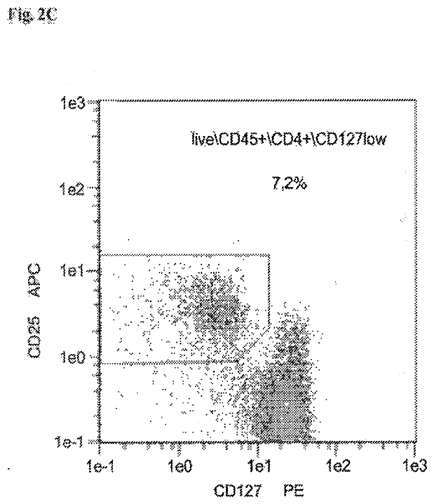

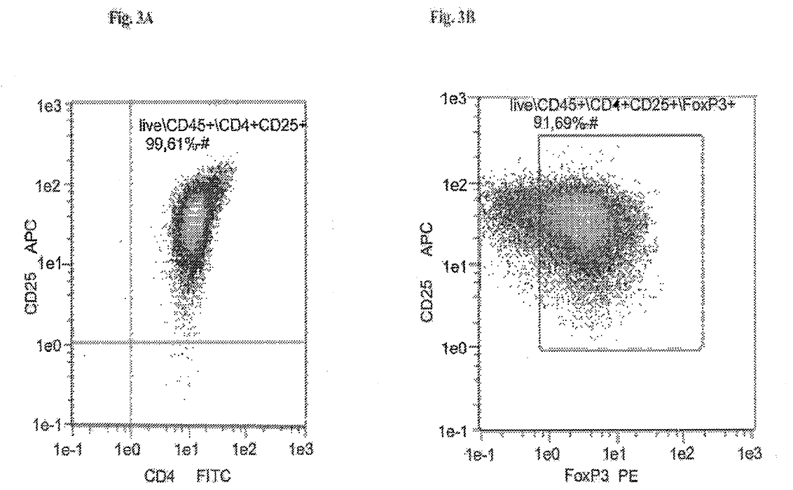

Expression of T cell markers on these cells is shown in FIGS. 2A-2C from one representative experiment (total 19): 97.5% of cells expressed CD4⁺, and 12.6% of these CD⁺cells co-expressed CD25⁺ (FIG. 2A); 6.3% of these CD4⁺ cells co-expressed Foxp3⁺ (FIG. 2B); and 7.2% of these CD4⁺ cells co-expressed CD127^{low} (FIG. 2C).

D. Modification and Expasion of CD4⁺CD25⁺Foxp3⁺CD127^{low} T Reg Cells

The medium used for the T reg cell culture was RPMI-1640 which contains phenol red, L-glutamine, and 25 MM HEPES (Gibco, UK) with the addition of both 5-10% autologous serum and 1% pen/strep (Gibco, UK). This medium was supplemented with 1 ng/ml-50 ng/ml transforming growth factor 1 (TGF 1) (R&D Systems, UK), 10 U/ml-1000 U/ml interleukin-2, (IL-2, R&D Systems, UK), 0.1 μg/ml-10 μg/ml mouse anti-human CD3 mAbs (Med biospecter, RF), and 0.1 μg/ml-10 μg/ml mouse anti-human CD28 mAbs (BD Pharmingen, USA). The expanded CD4⁺ T cells were cultured at 37° C. in 5% CO₂ for 6 to 8 days in flasks (either 25 cm² or 75 cm²) with all supplements. After 3 to 4 days, IL-2, TGFB1, anti-human CD3mAbs, and anti-human CD28 mAbs were added.

E. Phenotypic Characterizations of T Reg Cells After Expansion ex vivo

Autologous, modified and expanded cells were characterized at the end of culture. Flow cytometry was used to estimate the total numbers of live cells and the proportion of CD4⁺CD25⁺Foxp3⁺CD27^{low} cells in the cell suspension. To assure that the endotoxin levels in cell preparations were negligible, aliquots were tested with the Limulus assay kit (Sigma-Aldrich, USA), according to the manufacturer's protocols.

Table 1 shows the results of flow cytometry of initial CD4⁺ T cells and the same cells after 6 to 7 days of culture with stimulating molecules.

TABLE 1

| | | Marker Measurement in |
|---|---|---|
| | Marker Measurement | Modified and |
| Markers | in Initial cells | Expanded cells |
| CD4⁺ | 93.9 ± 4.5 | 99.8 ± 0.2 |
| CD4⁺CD25^{hi} | 15.7 ± 4.0 | 95.9 ± 2.4 |
| CD4⁺CD25⁺CD62L⁺ | 18.8 ± 9.0 | 54.6 ± 3.8 |
| CD4⁺CD25⁺Foxp3⁺ | 6.1 ± 4.8 | 89.6 ± 3.2 |
| CD4⁺CD25⁺CD152⁺ | 5.4 ± 2.7 | 93.8 ± 3.0 |
| CD4⁺CD25⁺CD127^{low} | 6.7 ± 4.1 | 91.3 ± 3.2 |

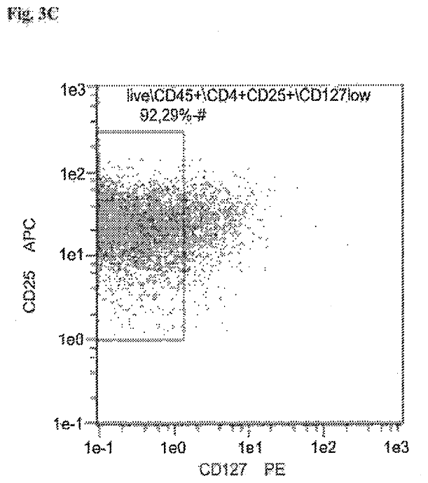

FIGS. 3A-3C show a representative sample of T reg cells expression after 6 days of ex vivo culture. 99.6% CD4⁺ T cells co-expressed CD25^{hi} (FIG. 3A); 91.7% CD4⁺ T cells co expressed Foxp3 (FIG. 3B); and 92.3% CD4⁺ cells co-expressed CD127^{low} (FIG. 3C).

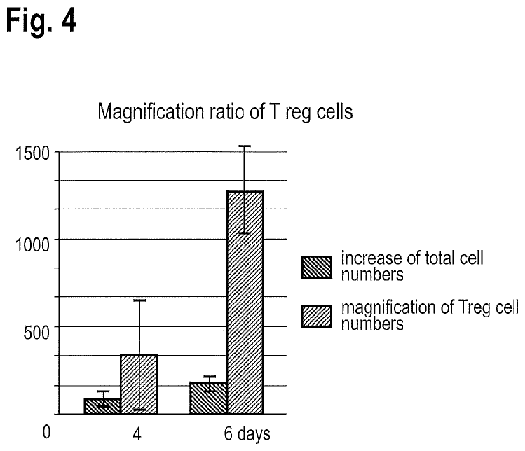

During the 6 days of propagating CD4 T cells (obtained from 19 donors), the total amount of cells increased 27.2±7.3 X, whereas the number of T reg cells CD4⁺CD25⁺Foxp3⁺ increased 1272±470 X (FIG. 4).

### F. Functional Capacity of Modified and Expanded T Reg Cells

To determine if ex vivo-modified and expanded T reg cells keep their own suppressive ability, their suppressive capacity to inhibit proliferation of target cells in a mixed lymphocyte reaction (MLR) was compared initially and after the expansion of T reg cells.

To this end, autologous target cells (CD4⁺, CD4⁺CD25) were isolated using the magnetic beads selection method (Miltenyi Biotec), stained with carboxyfluorescein succinimidyl ester (CFSE, Fluka, USA), and cultivated with or without equal numbers (1:1) of native T reg cells isolated from human blood or induced, ex vivo-expanded T reg cells. Either T cells CD4⁺CD2S⁻ T cells or CD4⁺ T cells were stimulated by 5 μg/ml (CD3 mAbs and allogeneic MNC treated with mitomycin C and depleted of CD3+ T cells by the magnetic bead selection method (Miltenyi Biotec).

After 4 to 5 days of culture, cell proliferation was estimated by measurement of a reduction of 5(6) Carboxyfluorescein diacetate N-succinimidyl ester (CFSE) in proliferating cells.

The functional activity of T reg cells isolated from the peripheral blood of GvHD patients is found to be substantially reduced.

This, it has been determined that the number of T reg cells in a patient suffering from GvHD is variable. This was determined by studying the immuno-phenotype of these cells within one group of patients in both the relapse stage.

### Example 2

### Treatment of GvHD with T Reg Cells

Five transplant patients with a reduced number of T reg cells in their peripherical blood were treated with autologous, ex vivo- modified and expanded CD4⁺CD25⁺CD127^{low} T reg cells.

Patients undergoing treatment did not receive either steroids or immunotherapy for at least 3 consecutive months. All the patients signed Consent Agreement before taking part in the study.

It is expected that treated patients do not suffer GvHD or have much reduced symptoms where the number of T reg cells in their peripherical blood increase treatment.

Treatment with autologous, ex vivo-modified and expanded T reg cells starting one or two weeks after transplant increases the amount of GvHD.

### Equivalents

Those skilled in the art will recognize, or be able to ascertain, using no more than routine experimentation, numerous equivalents to the specific embodiments described specifically herein. Such equivalents are intended to be encompassed in the scope of the following claims.

1. A method of treating and preventing graft-versus host disease (GvHD) in a mammalian transplant patient, comprising administration to the patient pharmaceutical formulation comprising:
a therapeutically effective amount of autologous, ex vivo-modified and expanded, autologous regulatory CD4⁺CD25⁺Foxp⁺CD127^{low} T cells (T reg); and
a pharmaceutically acceptable carrier.

2. The method of claim 1, wherein the T reg cells have been derived from the peripheral blood of the subject to be treated.

3. The method of claim 1, wherein the T reg cells have been stimulated with a CD3 mAb, aCD28 mAb, TGF- 1, and IL-2.

4. The method of claim 1, wherein the T reg cells are present at 1×10⁶ to 1.1×10⁷ T reg cell/kg body weight of the subject to be treated.

5. The method of claim 4, wherein the T reg cells are present at 2×10⁶ to 8×10⁶ T reg cells/kg body weight of the subject to be treated.

6. The method of claim 4, wherein the T reg cells are present at 4×10⁶ to 7×10⁶ T reg cells/kg body weight of the subject to be treated.

7. The method of claim 4, wherein the T reg cells are present at 5×10⁶ to 7×10⁶ T reg cells/kg body weight of the subject to be treated.

8. The method of claim 1, wherein the carrier is an intravenous glucose/dextrose solution, Ringer's lactate solution, sodium lactate solution, or Rheopolyglukin.

9. The method of claim 1, further comprising another compound which treats GvH D.
